(19) United States
(12) Patent Application Publication
(10) Pub. No.: US 2024/0099586 A1
(43) Pub. Date: Mar. 28, 2024

(54) ANALYSIS DEVICE AND ANALYSIS METHOD

(71) Applicants: HAMAMATSU PHOTONICS K.K., Hamamatsu-shi, Shizuoka (JP); TOKYO MEDICAL UNIVERSITY, Shinjuku-ku, Tokyo (JP)

(72) Inventors: Tomomi IIDA, Hamamatsu-shi, Shizuoka (JP); Yukio UEDA, Hamamatsu-shi, Shizuoka (JP); Takafumi HAMAOKA, Shinjuku-ku, Tokyo (JP)

(73) Assignees: HAMAMATSU PHOTONICS K.K., Hamamatsu-shi, Shizuoka (JP); TOKYO MEDICAL UNIVERSITY, Shinjuku-ku, Tokyo (JP)

(21) Appl. No.: 18/266,355

(22) PCT Filed: Dec. 16, 2021
(86) PCT No.: PCT/JP2021/046553

§ 371 Date: Jun. 9, 2023

(30) Foreign Application Priority Data
Jan. 18, 2021 (JP) 2021-005585

(51) Int. Cl. A61B 5/00; G01J 3/42; G01N 21/3563

(52) U.S. Cl. CPC A61B 5/0075; A61B 5/4872; A61B 5/7203; G01J 3/42; G01N 21/3563; G01J 2003/2859

(57) ABSTRACT

An analysis apparatus includes a light emission unit configured to emit measurement light including light in a 900 nm wavelength band to a sample, a light detection unit configured to acquire spectrum data of reflected light in the sample, a data processing unit configured to perform a noise removing process on the spectrum data, a first determination unit configured to store a PLS regression model associated with prediction of an amount of triglyceride in the sample and to determine an amount of triglyceride in the sample by applying the spectrum data subjected to the noise removing process to the PLS regression model, and a second determination unit configured to store data indicating a correlation with an amount of triglyceride in the sample and to determine an amount of brown adipose tissue or beige fat in the sample based on the data and the amount of triglyceride determined by the first determination unit.

### TECHNICAL FIELD

The present disclosure relates to an analysis apparatus and an analysis method.

### BACKGROUND ART

Brown adipose tissue (BAT) is adipose tissue and also has the features that it burns fat and dissipates energy by specific uncoupling protein 1 (UCP1). In recent studies, it has been reported that brown adipose tissue affects the whole body metabolism of glucose and lipid, and insulin sensitivity. It has also been reported that brown adipose tissue is associated with whole body metabolism control via various brown adipose tissue-derived transmitters and nerves and plays a role as an endocrine organ. When the amount or activity of brown adipose tissue can be controlled, prevention or improvement of metabolic diseases such as metabolic syndrome can be expected.

To date, as means for measuring brown adipose tissue in humans, ¹⁸F-fluorodeoxyglucose-positron emission tomography (FDG-PET) has been widely used. For example, a measuring method described in Patent Literature 1 is known as a noninvasive method of measuring human brown adipose tissue. In this method, brown adipose tissue is evaluated based on total hemoglobin concentration at measurement region using near-infrared time-resolved spectroscopy (TRS).

Recently, it has become clear that human brown adipose tissue is largely composed of beige adipocytes. Beige adipocytes are white adipocytes with UCP1 appearing in it, turning it beige and having properties similar to those of brown adipose tissue. For example, methods described in Non-Patent Literature 1 and 2 are known as a noninvasive method of measuring beige fat. These methods are both based on a reflectance spectrum of a measuring region.

Non-Patent Literature 1 discloses that beige-ing of white adipose tissue (WAT) is detected based on a reflectance intensity ratio at wavelengths 550 nm and 680 nm and a slope of a spectrum at wavelengths of 570 nm to 630 nm. Non-Patent Literature 2 discloses that volume fractions of lipid and water are determined such that a difference between a measured diffuse reflectance spectrum acquired in a wavelength range of 1050 nm to 1350 nm and a reflectance spectrum modeled from a lookup table based on Monte-Carlo simulation is minimized, and beige-ing of white adipose tissue is detected from the volume fractions.

### CITATION LIST

### Patent Literature

[Patent Literature 1] Japanese Patent No. 6224464
[Non-Patent Literature 1] “Diffuse optical spectroscopy and imaging to detect and quantify adipose tissue browning” U.S Dinish et al., Scientific Reports, vol. 7, 41357 (2017)
[Non-Patent Literature 2] “Quantitative in vivo detection of adipose tissue browning using diffuse reflectance spectroscopy in near-infrared II window” Kapil Dev et al., Journal of Biophotonics, Volume 11, Issue 12e201800135, December (2018)

### SUMMARY OF INVENTION

### Technical Problem

In order to promote study of human brown adipose tissue and clinical applications such as treatment and disease prevention using brown adipose tissue, there is demand for methods dedicated for analysis of brown adipose tissue and beige fat. For example, if quantitative evaluation of brown adipose tissue or beige fat as well as relative evaluation of brown adipose tissue or beige fat could be performed, it is thought that this would become the cornerstone for study and application of brown adipose tissue in the future.

The present disclosure was invented to solve the aforementioned problems, and an objective thereof is to provide an analysis apparatus and an analysis method that can enable quantitative evaluation of brown adipose tissue or beige fat.

### Solution to Problem

The applicant of the present disclosure paid attention to spectral characteristics of brown adipose tissue and beige fat while repeatedly carrying out earnest research of the aforementioned problems, and found that brown adipose tissue and beige fat have optical absorption characteristics which has not been seen in white fat. When cold stimulation was applied to brown adipose tissue and beige fat, the optical absorption characteristics of brown adipose tissue and beige fat changed along with the cold stimulation. It was also seen that there is a predetermined correlation between an amount of change of the optical absorption characteristics and an amount of change of triglyceride obtained from the biochemical assay. Therefore, the applicant of the present disclosure obtained knowledge that quantitative evaluation of brown adipose tissue or beige fat can be simply carried out by combining analysis of an amount of triglyceride based on spectrum data of a sample and a regression model associated with prediction of an amount of triglyceride in the sample, and completed details of the present disclosure as a result.

An analysis apparatus according to an aspect of the present disclosure includes: a light emission unit configured to emit measurement light including light in a 900 nm wavelength band to a sample; a light detection unit configured to detect reflected light from the sample and to acquire spectrum data of the reflected light in the sample; a data processing unit configured to perform a noise removing process on the spectrum data acquired by the light detection unit; a first determination unit configured to store a PLS (Partial Least Square) regression model associated with prediction of an amount of triglyceride in a sample and to determine an amount of triglyceride in the sample by applying the spectrum data subjected to the noise removing process to the PLS regression model; and a second determination unit configured to store data indicating a correlation with an amount of triglyceride in a sample and to determine an amount of brown adipose tissue or beige fat in the sample based on the data and the amount of triglyceride determined by the first determination unit.

In this analysis apparatus, spectrum data of reflected light from a sample is acquired, and a noise removing process is performed on the acquired spectrum data. In the spectrum data subjected to the noise removing process, absorption characteristics of light in a 900 nm wavelength band vary depending on whether brown adipose tissue, beige fat, and white fat are present in the sample. Accordingly, by applying the spectrum data subjected to the noise removing process to the PLS regression model prepared in advance, it is possible to determine an amount of triglyceride in the sample. By comparing the result of determination of an amount of triglyceride with data indicating a correlation with the amount of triglyceride in the sample, it is possible to determine an absolute value of an amount of brown adipose tissue or beige fat in the sample.

The PLS regression model may be a model based on an intensity of lipid absorption peak in the spectrum data subjected to the noise removing process. It is possible to enhance determination accuracy of an amount of triglyceride in the sample by using the PLS regression model based on an intensity of lipid absorption peak.

The first determination unit may determine whether brown adipose tissue, beige fat, and white fat are present in the sample based on whether there is a water absorption peak in a 900 nm wavelength band in the spectrum data subjected to the noise removing process. In brown adipose tissue and beige fat, a water absorption peak is likely to appear in the 900 nm wavelength band. In white fat, a water absorption peak is not likely to appear in the 900 nm wavelength band. By determining whether brown adipose tissue, beige fat, and white fat are present in the sample based on whether there is a water absorption peak in the 900 nm wavelength band, it is possible to further increase an amount of information acquired through analysis.

The second determination unit may store data indicating a correlation with an amount of triglyceride in a stimulated sample and determine an amount of brown adipose tissue or beige fat in the stimulated sample based on the data and the amount of triglyceride determined by the first determination unit. In this case, it is possible to accurately determine an amount of brown adipose tissue or beige fat in the stimulated sample. Since change of the amount of brown adipose tissue or beige fat in the sample before and after stimulation has been applied thereto can be reliably determined, it is possible to further broaden applications of the analysis.

An analysis method according to another aspect of the present disclosure includes: a light emission step of emitting measurement light including light in a 900 nm wavelength band to a sample; a light detection step of detecting reflected light from the sample and acquiring spectrum data of the reflected light in the sample; a data processing step of performing a noise removing process on the spectrum data acquired in the light detection step; a first determination step of using a PLS regression model associated with prediction of an amount of triglyceride and determining an amount of triglyceride in the sample by applying the spectrum data subjected to the noise removing process to the PLS regression model; and a second determination step of using data indicating a correlation with an amount of triglyceride in a sample and determining an amount of brown adipose tissue or beige fat in the sample based on the data and the amount of triglyceride determined in the first determination step.

In this analysis method, spectrum data of reflected light from a sample is acquired, and a noise removing process is performed on the acquired spectrum data. In the spectrum data subjected to the noise removing process, absorption characteristics of light in a 900 nm wavelength band vary depending on whether brown adipose tissue, beige fat, and white fat are present in the sample. Accordingly, by applying the spectrum data subjected to the noise removing process to the PLS regression model prepared in advance, it is possible to determine an amount of triglyceride in the sample. By comparing the result of determination of an amount of triglyceride with data indicating a correlation with the amount of triglyceride in the sample, it is possible to determine an absolute value of an amount of brown adipose tissue or beige fat in the sample.

The PLS regression model may be a model based on an intensity of lipid absorption peak in the spectrum data subjected to the noise removing process. It is possible to enhance determination accuracy of an amount of triglyceride in the sample by using the PLS regression model based on an intensity of lipid absorption peak.

The first determination step may include determining whether brown adipose tissue, beige fat, and white fat are present in the sample based on whether there is a water absorption peak in a 900 nm wavelength band in the spectrum data subjected to the noise removing process. In brown adipose tissue and beige fat, a water absorption peak is likely to appear in the 900 nm wavelength band. In white fat, a water absorption peak is not likely to appear in the 900 nm wavelength band. By determining whether brown adipose tissue, beige fat, and white fat are present in the sample based on whether there is a water absorption peak in the 900 nm wavelength band, it is possible to further increase an amount of information acquired through analysis.

The second determination step may include using data indicating a correlation with an amount of triglyceride in a stimulated sample and determining an amount of brown adipose tissue or beige fat in the stimulated sample based on the data and the amount of triglyceride determined in the first determination step. In this case, it is possible to accurately determine an amount of brown adipose tissue or beige fat in the stimulated sample. Since change of the amount of brown adipose tissue or beige fat in the sample before and after stimulation has been applied thereto can be reliably determined, it is possible to further broaden applications of the analysis.

### Advantageous Effects of Invention

According to the present disclosure, it is possible to enable quantitative evaluation of brown adipose tissue or beige fat.

### BRIEF DESCRIPTION OF DRAWINGS

FIG. 1 is a block diagram illustrating a configuration of an analysis apparatus according to an embodiment of the present disclosure.

FIG. 2 is a diagram illustrating examples of reflected light in control groups.

FIG. 3 is a diagram illustrating examples of reflected light in stimulated groups.

FIG. 4 is a diagram illustrating examples of predicted values of amounts of triglyceride in brown adipose tissue from PLS regression model.

FIG. 5 is a diagram illustrating examples of predicted values of amounts of triglyceride in beige fat from the PLS regression model.

FIG. 6 is a diagram illustrating correlation between amounts of triglyceride and amounts of brown adipose tissue in control group.

FIG. 7 is a diagram illustrating correlation between amounts of triglyceride and amounts of beige fat in control group.

FIG. 8 is a diagram illustrating correlation between amounts of triglyceride and amounts of brown adipose tissue in stimulated group.

FIG. 9 is a diagram illustrating correlation between amounts of triglyceride and amounts of beige fat in stimulated group.

FIG. 10 is a flowchart illustrating an analysis method according to an embodiment of the present disclosure.

FIG. 11(a) is a diagram illustrating time-course changes of lipid absorption peak in brown adipose tissue in control groups and stimulated groups, and FIG. 11(b) is a diagram illustrating time-course changes of amounts of triglyceride in brown adipose tissue in control groups and stimulated groups.

FIG. 12(a) is a diagram illustrating time-course changes of lipid absorption peak in beige fat in control groups and stimulated groups, and FIG. 12(b) is a diagram illustrating time-course changes of amounts of triglyceride in beige fat in control groups and stimulated groups.

### DESCRIPTION OF EMBODIMENTS

Hereinafter, exemplary embodiments of an analysis apparatus and an analysis method according to an aspect of the present disclosure will be described in detail with reference to the accompanying drawings.

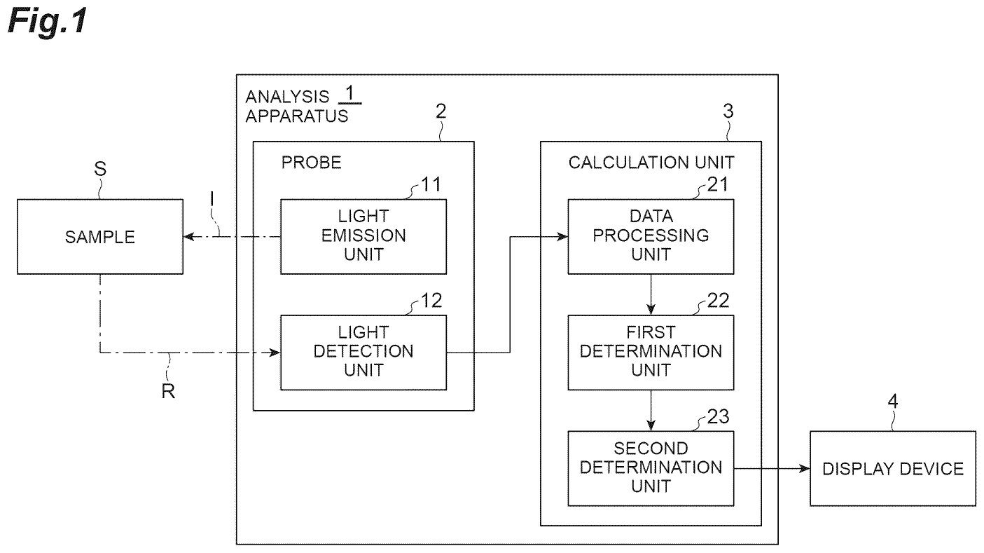

FIG. 1 is a block diagram illustrating a configuration of an analysis apparatus according to an embodiment of the present disclosure. The analysis apparatus 1 illustrated in FIG. 1 is configured as an apparatus that measures an absolute value of an amount of brown adipose tissue or an absolute value of an amount of beige fat in a sample S. The analysis apparatus 1 serves to enable determination of brown adipose tissue and beige fat and white fat which was difficult using positron emission tomography (PET) inspection, thermography, or the like according to the related art and to simply implement quantitative evaluation of brown adipose tissue or beige fat. The sample S is, for example, biological tissue of humans or animals. The sample S may be tissue in vivo or excised tissue.

Brown adipose tissue is adipose tissue and also has features of burning fat and dissipating energy by specific uncoupling protein 1 (UCP1). A cell origin thereof is a dermomyotomal precursor. Morphological characteristics thereof include multilocular lipid droplets and abundant mitochondria. Human brown adipose tissue is mainly present in the interscapular regions in infants and perirenal region in an adult. Beige fat is beige-ing with induction of UCP1 in white fat and which has the same features as brown adipose tissue. Human beige fat is mainly present in the supraclavicular and paravertebral regions. A cell origin thereof is a preadipose cell. Morphological features thereof include multilocular lipid droplets and abundant mitochondria similarly to the brown adipose tissue. White adipose tissue mainly has storage and discharge of energy as physiological functions. Human white adipose tissue is mainly present subcutaneously or around internal organs. A cell origin thereof is a preadipose cell. Morphological features thereof include unilocular lipid droplets. A major component of white adipose tissue is triglyceride.

The analysis apparatus 1 can be applied, for example, to the field of medicine or the field of sports. In the field of medicine, application to treatment and prevention of a diabetic or a lipid metabolism disorder is conceivable. It is known that brown adipose tissue is highly associated with insulin sensitivity or lipid metabolism. By comparing an amount of brown adipose tissue or beige fat of a diabetic patient or a patient with a lipid metabolism disorder with an amount of brown adipose tissue or beige fat of a healthy subject and increasing the amount of brown adipose tissue or beige fat, treatment and prevention not depending on medicine is expected.

Prevention or improvement of metabolic diseases such as metabolic syndrome is achieved by measuring an amount of brown adipose tissue or beige fat. By adding data of an amount of brown adipose tissue or beige fat to examination which is carried out in schools or the like, prevention of diseases or weight control for young people becomes possible. By investigating the relationship between an increase in visceral fat and an amount of brown adipose tissue or beige fat after middle-aged, advancement of research on aging can be expected. It has been reported that a mouse with a reinforced function of brown adipose tissue lives longer. It is also conceivable that the present disclosure be applied to evaluation means in developing supplements or the like for prompting an increase or activation of brown adipose tissue or beige fat.

In the field of sports, for example, evaluation of a training program or provision of a new weight loss program becomes possible by measuring an amount of brown adipose tissue or beige fat before and after training. When weight control for reducing only fat without losing muscle mass is necessary, an approach based on an increase of the amount of brown adipose tissue or beige fat is conceivable as a third option in addition to diet and training Monitoring of the amount of brown adipose tissue or beige fat is expected to help weight control.

A configuration of the analysis apparatus 1 will be described below. As illustrated in FIG. 1, the analysis apparatus 1 includes a probe 2 and a calculation unit 3. The analysis apparatus 1 is communicatively connected to a display device 4. The display device 4 is, for example, a monitor or a touch panel display. The display device 4 receives analysis result information from the analysis apparatus 1 and displays the received information.

The probe 2 includes a light emission unit 11 and a light detection unit 12. The probe 2 is communicatively connected to the calculation unit 3. The probe 2 may be a handy probe in which the light emission unit 11 and the light detection unit 12 are accommodated in a small casing. The probe 2 may be a wireless probe that can communicate with the calculation unit 3 in a wireless manner. In this case, it is possible to enhance flexibility of a measuring posture in the analysis apparatus 1. For example, by fixing the wireless probe to a part of a body, measurement while eating or training is possible and acquisition of data of time-course change in a day is also facilitated.

The light emission unit 11 is a unit that emits measurement light I including light in a 900 nm wavelength band to a sample S. For example, a halogen light source, an LD, an LED, or an SLD can be used as a light source of the light emission unit 11. The wavelength band of measurement light I ranges, for example, from 900 nm to 1000 nm. This wavelength band includes 920 nm to 930 nm in which lipid absorption peak appears and 960 nm to 970 nm in which a water absorption peak appears.

The light detection unit 12 is a unit that detects reflected light R from the sample S and acquires spectrum data of the reflected light R in the sample S. For example, a CCD array, a CMOS array, or a PD array can be used as a detection element of the light detection unit 12. The light detection unit 12 outputs information indicating a detection result to the calculation unit 3. The light detection unit 12 may include an integrating sphere. In this case, the light detection unit 12 can acquire uniform diffuse reflectance spectrum data of the reflected light R by causing the reflected light R from the sample S to be diffused and reflected in the integrating sphere.

The calculation unit 3 is a unit that performs various types of calculation based on information from the light detection unit 12. The calculation unit 3 is physically a computer system including a memory such as a RAM or a ROM, a processor (an operation circuit) such as a CPU, a communication interface, and a storage unit such as a hard disk. Examples of the computer system include a personal computer, a cloud server, a smart device (such as a smartphone or a table terminal). The calculation unit 3 serves as a controller of the analysis apparatus 1 by causing the CPU of the computer system to execute a program stored in the memory.

The calculation unit 3 includes a data processing unit 21, a first determination unit 22, and a second determination unit 23 as functional elements. The data processing unit 21 is a unit that performs a noise removing process on the spectrum data acquired by the light detection unit 12. An example of the noise removing process is second-order derivative such as Savitzky-Golay smoothing. The data processing unit 21 outputs the spectrum data subjected to the noise removing process to the first determination unit 22.

The first determination unit 22 is a unit that determines an amount of triglyceride in the sample S. When the spectrum data subjected to the noise removing process is received from the data processing unit 21, the first determination unit 22 determines whether brown adipose tissue, beige fat, and white fat are present in the sample S based on whether a water absorption peak in the 900 nm wavelength band is present in the spectrum data.

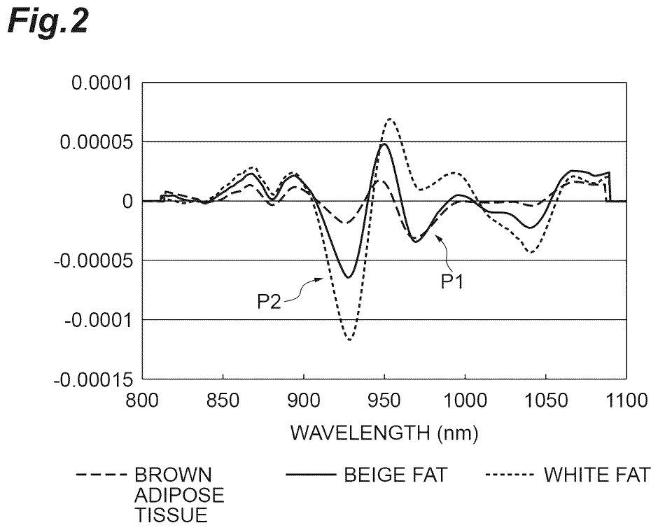

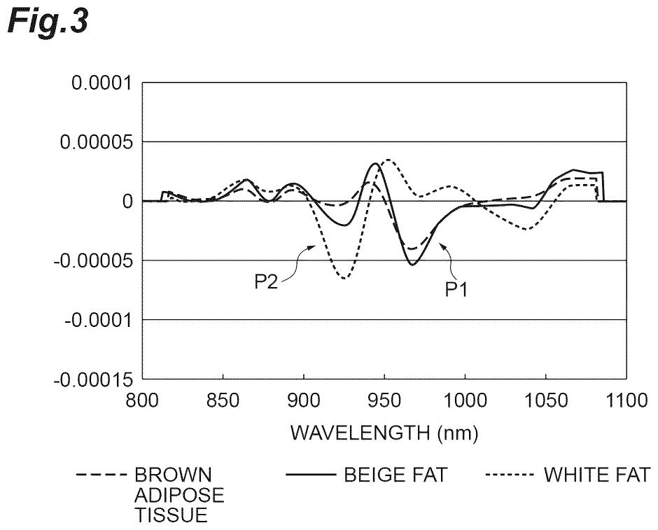

FIGS. 2 and 3 are diagrams illustrating examples of reflectance spectra. FIG. 2 illustrates second-derivative spectra in unstimulated sample groups (control groups). FIG. 3 illustrates second-derivative spectra in stimulated sample groups (stimulated groups). Here, the stimulation is cold stimulation. In the control group, five rats raised at the room temperature of 24° C. for 28 days were prepared. In the stimulated group, five rats raised at the room temperature of 4° C. for 28 days were prepared.

It can be seen from the results illustrated in FIGS. 2 and 3 that a water absorption peaks (peaks with a negative value) P1 are present at the wavelength near 960 nm to 970 nm in brown adipose tissue and beige fat regardless of stimulation. It can be seen from the results illustrated in FIGS. 2 and 3 that water absorption peaks (peaks with a negative value) P1 are not present at the wavelength near 960 nm to 970 nm in white fat regardless of stimulation. It can be seen from these results that whether brown adipose tissue, beige fat, and white fat are present in the sample S can be determined based on whether water absorption peaks in the 900 nm wavelength band are present in the spectra data.

The first determination unit 22 stores a PLS regression model associated with prediction of an amount of triglyceride in the sample S in determining the amount of triglyceride in the sample S. The PLS regression model is a model based on an intensity of lipid absorption peak in the spectrum data subjected to the noise removing process. When the spectrum data subjected to the noise removing process is received from the data processing unit 21, the first determination unit 22 determines the amount of triglyceride in the sample S by applying the spectrum data to the PLS regression model. The first determination unit 22 generates information indicating the determination result of the amount of triglyceride in the sample S and outputs the generated information to the second determination unit 23.

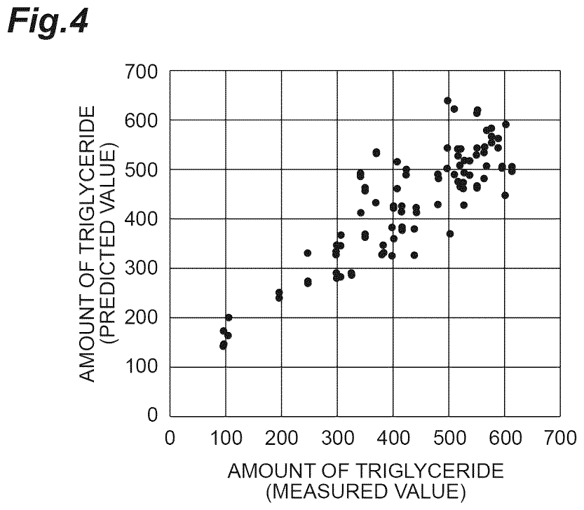

FIG. 4 is a diagram illustrating examples of predicted values of amounts of triglyceride in brown adipose tissue from the PLS regression model. In the drawing, the horizontal axis represents the measured value of the amount of triglyceride, and the vertical axis represents the predicted value of the amount of triglyceride. A relationship between the amount of triglyceride in brown adipose tissue predicted through PLS regression and the actual amount of triglyceride in brown adipose tissue can be ascertained from the result illustrated in FIG. 4. In FIG. 4, a determination coefficient for calibration (model preparation data) is R²=0.75, and a determination coefficient for validation (verification data) is R²=0.73.

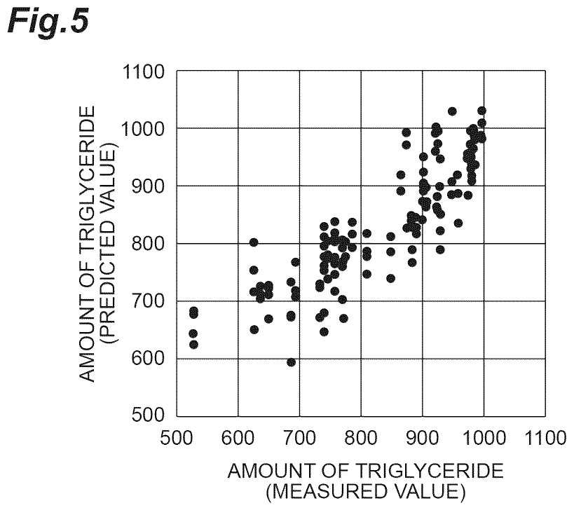

FIG. 5 is a diagram illustrating examples of predicted values of amounts of triglyceride in beige fat from the PLS regression model. In the drawing, the horizontal axis represents the measured value of the amount of triglyceride, and the vertical axis represents the predicted value of the amount of triglyceride. A relationship between the amount of triglyceride in beige fat predicted through PLS regression and the actual amount of triglyceride in beige fat can be ascertained from the result illustrated in FIG. 5. In FIG. 5, a determination coefficient for calibration (model preparation data) is R²=0.73, and a determination coefficient for validation (verification data) is R²=0.53.

The second determination unit 23 is a unit that determines an amount of brown adipose tissue or beige fat in the sample S. The second determination unit 23 stores data indicating a correlation with an amount of triglyceride in a sample S in determining the amount of brown adipose tissue or beige fat in the sample S. When the information indicating the determination result of the amount of triglyceride in the sample S is received from the first determination unit 22, the second determination unit 23 determines the amount of brown adipose tissue or beige fat in the sample S based on the data and the amount of triglyceride determined by the first determination unit 22. The second determination unit 23 generates information indicating the determination result of the amount of brown adipose tissue or beige fat in the sample S and outputs the generated information to the display device 4.

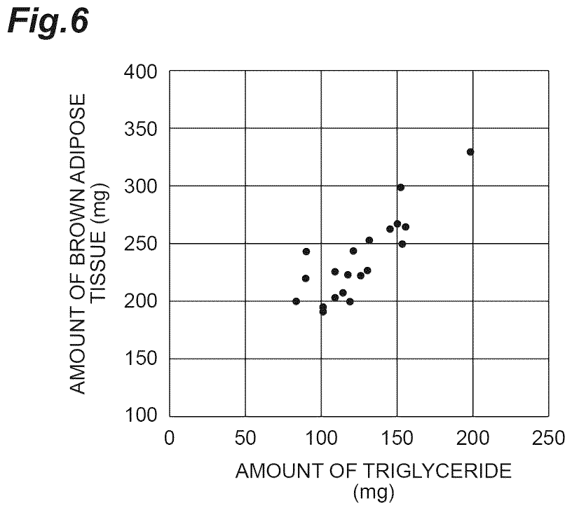

FIG. 6 is a diagram illustrating the correlation between amounts of triglyceride and amounts of brown adipose tissue in the control group. In the drawing, the horizontal axis represents the amount of triglyceride, and the vertical axis represents the amount of brown adipose tissue. It can be seen from the result illustrated in FIG. 6 that there is the predetermined correlation between the amount of triglyceride and the amount of brown adipose tissue in the control group. A determination coefficient for the correlation in the drawing is R²=0.72. An absolute value of the amount of brown adipose tissue can be determined based on the amount of triglyceride determined by the first determination unit 22 by referring to the correlation.

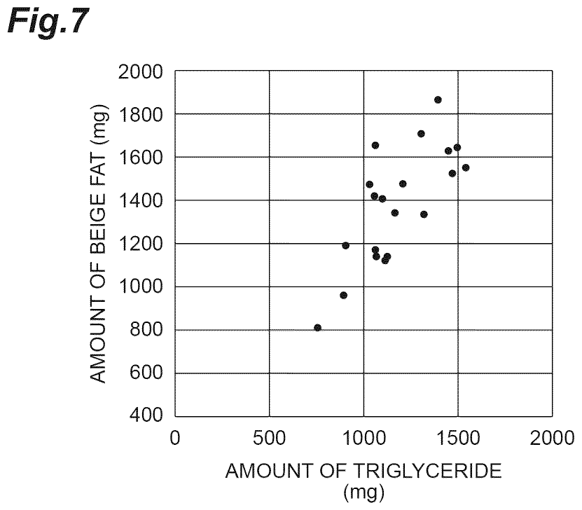

FIG. 7 is a diagram illustrating the correlation between amounts of triglyceride and amounts of beige fat in the control group. In the drawing, the horizontal axis represents the amount of triglyceride, and the vertical axis represents the amount of beige fat. It can be seen from the result illustrated in FIG. 7 that there is the predetermined correlation between the amount of triglyceride and the amount of beige fat in the control group. A determination coefficient for the correlation in the drawing is R²=0.57. An absolute value of the amount of beige fat can be determined based on the amount of triglyceride determined by the first determination unit 22 by referring to the correlation.

The second determination unit 23 may store data indicating a correlation with triglyceride in a stimulated sample S. In this case, the second determination unit 23 determines the amount of brown adipose tissue or beige fat in the stimulated sample S based on the data and the amount of triglyceride determined by the first determination unit 22. The second determination unit 23 generates information indicating the determination result of the amount of brown adipose tissue or beige fat in the stimulated sample S and outputs the generated information to the display device 4. The second determination unit 23 may determine both the amount of brown adipose tissue and beige fat in the sample S or may determine only one thereof.

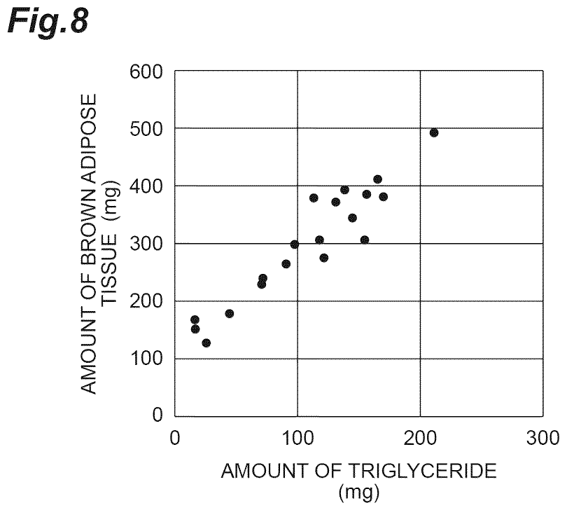

FIG. 8 is a diagram illustrating the correlation between amounts of triglyceride and amounts of brown adipose tissue in the stimulated group. In the drawing, the horizontal axis represents the amount of triglyceride, and the vertical axis represents the amount of brown adipose tissue. It can be seen from the result illustrated in FIG. 8 that there is the predetermined correlation between the amount of triglyceride and the amount of brown adipose tissue in the stimulated group. A determination coefficient for the correlation in the drawing is R²=0.9. An absolute value of the amount of brown adipose tissue can be determined based on the amount of triglyceride determined by the first determination unit 22 by referring to the correlation.

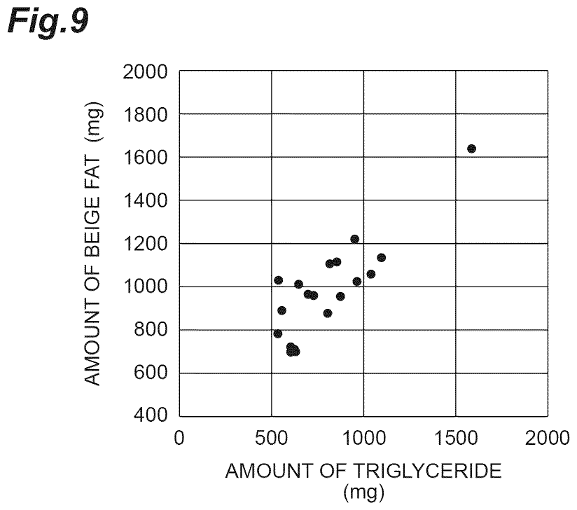

FIG. 9 is a diagram illustrating the correlation between amounts of triglyceride and amounts of beige fat in the stimulated group. In the drawing, the horizontal axis represents the amount of triglyceride, and the vertical axis represents the amount of beige fat. It can be seen from the result illustrated in FIG. 9 that there is the predetermined correlation between the amount of triglyceride and the amount of beige fat in the stimulated group. A determination coefficient for the correlation in the drawing is R²=0.73. An absolute value of the amount of beige fat can be determined based on the amount of triglyceride determined by the first determination unit 22 by referring to the correlation.

An analysis method according to an embodiment of the present disclosure will be described below.

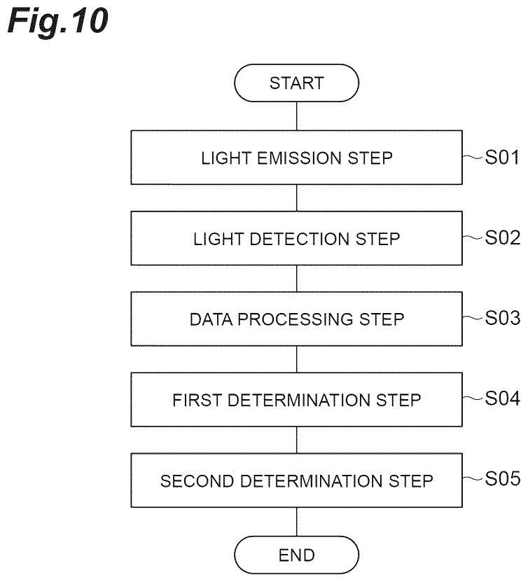

FIG. 10 is a flowchart illustrating the analysis method according to this embodiment. In this embodiment, for example, the analysis method using the analysis apparatus 1 is performed. As illustrated in FIG. 10, the analysis method includes a light emission step (Step S01), a light detection step (Step S02), a data processing step (Step S03), a first determination step (Step S04), and a second determination step (Step S05).

In the light emission step, the probe 2 of the analysis apparatus 1 is set in a sample S, measurement light I including light in the 900 nm wavelength band is emitted from the light emission unit 11 of the probe 2 to the sample S. The measurement light I emitted to the sample S is reflected by the sample S and becomes reflected light R. In the light detection step, the reflected light R from the sample S is detected by the light detection unit 12 of the probe 2 and spectrum data of the reflected light R in the sample S is acquired. When the light detection unit 12 includes the integrating sphere, the spectrum data acquired by the light detection unit 12 is uniform diffuse reflectance spectrum data of the reflected light R.

In the data processing step, the noise removing process is performed on the spectrum data acquired by the light detection unit 12. Here, as the noise removing process, differential processing such as second-order derivative or Savitzky-Golay smoothing is performed on the spectrum data acquired by the light detection unit 12.

In the first determination step, whether brown adipose tissue, beige fat, and white fat are present in the sample S is determined based on whether there is a water absorption peak in the 900 nm wavelength band in the spectrum data subjected to the noise removing process. In the first determination step, by using the PLS regression model associated with prediction of an amount of triglyceride and applying the spectrum data subjected to the noise removing process to the PLS regression model, the amount of triglyceride in the sample S is determined. A model based on an intensity of lipid absorption peak P2 in the spectrum data subjected to the noise removing process is used as the PLS regression model.

In the second determination step, data indicating a correlation with the amount of triglyceride in the sample S is used and the amount of brown adipose tissue or beige fat in the sample S is determined based on the data and the amount of triglyceride determined in the first determination step. When a sample S to which stimulation such as cold stimulation is applied is to be analyzed, data indicating a correlation with the amount of triglyceride in the stimulated sample S is used in the second determination step. In the second determination step, the amount of brown adipose tissue or beige fat in the stimulated sample S is determined based on the data and the amount of triglyceride determined by the first determination unit 22.

As described above, with the analysis apparatus 1 and the analysis method, spectrum data of reflected light R in the sample S is acquired, and the noise removing process is performed on the acquired spectrum data. In the spectrum data subjected to the noise removing process, absorption characteristics of light in the 900 nm wavelength band varies depending on whether brown adipose tissue, beige fat, and white fat are present in the sample S. Accordingly, by applying the spectrum data subjected to the noise removing process to a prepared PLS regression model, the amount of triglyceride in the sample S can be determined. By comparing the determination result of the amount of triglyceride with the data indicating the correlation with the amount of triglyceride in the sample S, the absolute value of the amount of brown adipose tissue or beige fat in the sample S can be determined.

In this embodiment, the PLS regression model is a model based on the intensity of the lipid absorption peak in the spectrum data subjected to the noise removing process. By using the PLS regression model based on the intensity of the lipid absorption peak P2, it is possible to enhance determination accuracy of the amount of triglyceride in the sample S.

In this embodiment, the first determination unit 22 determines whether brown adipose tissue, beige fat, and white fat are present in the sample S based on whether there is the water absorption peak P1 in the 900 nm wavelength band in the spectrum data subjected to the noise removing process. In brown adipose tissue and beige fat, the water absorption peak P1 is likely to appear in the 900 nm wavelength band. In white fat, the water absorption peak P1 is not likely to appear in the 900 nm wavelength band (see FIGS. 2 and 3). By determining whether brown adipose tissue, beige fat, and white fat are present in the sample S based on whether there is the water absorption peak in the 900 nm wavelength band, it is possible to obtain an analysis result associated with whether brown adipose tissue, beige fat, and white fat are present in addition to the absolute value of the amount of brown adipose tissue or beige fat. Accordingly, it is possible to further increase an amount of information acquired through analysis. For example, by not performing the process of determining the absolute value of the amount of brown adipose tissue or beige fat when it is determined that brown adipose tissue or beige fat is not present, it is possible to reduce processing time.

In this embodiment, the second determination unit 23 stores data indicating a correlation with the amount of triglyceride in the stimulated sample S and determines the amount of brown adipose tissue or beige fat in the stimulated sample S based on the data and the amount of triglyceride determined by the first determination unit 22. In this case, it is possible to accurately determine the amount of brown adipose tissue or beige fat in the stimulated sample S. Since change of the amount of brown adipose tissue or beige fat in the sample S before and after stimulation has been applied thereto can be reliably determined, it is possible to further broaden applications of the analysis.

The present disclosure is not limited to the aforementioned embodiment. For example, the analysis apparatus 1 may include a determination unit configured to determine an amount of change of brown adipose tissue or beige fat based on change of the intensity of the lipid absorption peak P2 (see FIGS. 2 and 3) in the spectrum data subjected to the noise removing process.

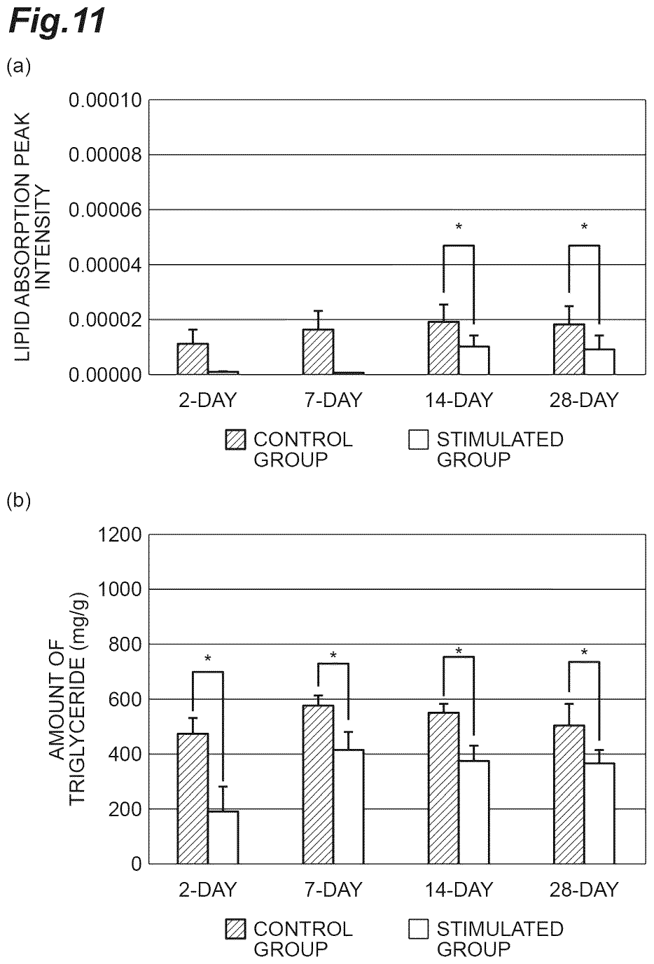

FIG. 11(a) is a diagram illustrating time-course changes of an intensity of lipid absorption peak in brown adipose tissue in the control groups and the stimulated groups. FIG. 11(b) is a diagram illustrating time-course changes of the amount of triglyceride in brown adipose tissue in the control groups and the stimulated groups. The results illustrated in the drawings were acquired based on biochemical assay. The lipid absorption peak in brown adipose tissue is present at wavelength near 924 nm.

It was ascertained from the result illustrated in FIG. 11(a) that the lipid absorption peak decreases significantly due to continuous cold stimulation in 14-day and 28-day data. It was ascertained from the result illustrated in FIG. 11(b) that the amount of triglyceride in brown adipose tissue in the stimulated groups is less than the amount of triglyceride in brown adipose tissue in the control groups in all periods. Accordingly, it was ascertained that the decrease of the lipid absorption peak exhibited a similar trend as the decrease of triglyceride acquired through biochemical assay.

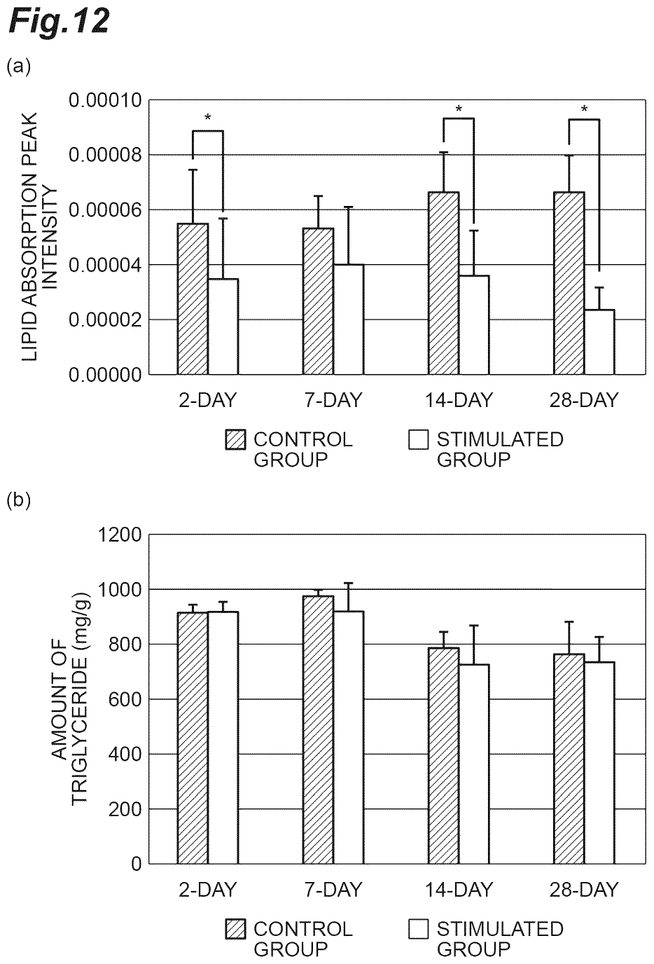

FIG. 12(a) is a diagram illustrating time-course changes of an intensity of lipid absorption peak in beige fat in the control groups and the stimulated groups. FIG. 12(b) is a diagram illustrating time-course changes of the amount of triglyceride in beige fat in the control groups and the stimulated groups. The results illustrated in the drawings were acquired based on biochemical assay. The lipid absorption peak in beige fat is present at wavelength near 926 nm.

It was ascertained from the result illustrated in FIG. 12(a) that the lipid absorption peak decreases significantly due to continuous cold stimulation in all data except 7-day data. It was ascertained from the result illustrated in FIG. 12(b) that the amount of triglyceride in beige fat in the stimulated groups is less than the amount of triglyceride in beige fat in the control groups in three data of 7-day, 14-day, and 28-day though the amount of decrease is less than that in the brown adipose tissue. Accordingly, it was ascertained that the decrease of the lipid absorption peak exhibited a similar trend as the decrease of triglyceride acquired through biochemical assay.

### REFERENCE SIGNS LIST

1 Analysis apparatus
11 Light emission unit
12 Light detection unit
21 Data processing unit
22 First determination unit
23 Second determination unit
L Measurement light
R Reflected light
S Sample
P1 Water absorption peak
P2 Lipid absorption peak

1: An analysis apparatus comprising:
a light emission unit configured to emit measurement light including light in a 900 nm wavelength band to a sample;
a light detection unit configured to detect reflected light from the sample and to acquire spectrum data of the reflected light in the sample;
a data processing unit configured to perform a noise removing process on the spectrum data acquired by the light detection unit;
a first determination unit configured to store a PLS regression model associated with prediction of an amount of triglyceride in a sample and to determine an amount of triglyceride in the sample by applying the spectrum data subjected to the noise removing process to the PLS regression model; and
a second determination unit configured to store data indicating a correlation with an amount of triglyceride in a sample and to determine an amount of brown adipose tissue or beige fat in the sample based on the data and the amount of triglyceride determined by the first determination unit.

2: The analysis apparatus according to claim 1, wherein the PLS regression model is a model based on an intensity of lipid absorption peak in the spectrum data subjected to the noise removing process.

3: The analysis apparatus according to claim 1, wherein the first determination unit determines whether brown adipose tissue, beige fat, and white fat are present in the sample based on whether there is a water absorption peak in a 900 nm wavelength band in the spectrum data subjected to the noise removing process.

4: The analysis apparatus according to claim 1, wherein the second determination unit stores data indicating a correlation with an amount of triglyceride in a stimulated sample and determines an amount of brown adipose tissue or beige fat in the stimulated sample based on the data and the amount of triglyceride determined by the first determination unit.

5: An analysis method comprising:
a light emission step of emitting measurement light including light in a 900 nm wavelength band to a sample;
a light detection step of detecting reflected light from the sample and acquiring spectrum data of the reflected light in the sample;
a data processing step of performing a noise removing process on the spectrum data acquired in the light detection step;
a first determination step of using a PLS regression model associated with prediction of an amount of triglyceride and determining an amount of triglyceride in the sample by applying the spectrum data subjected to the noise removing process to the PLS regression model; and
a second determination step of using data indicating a correlation with an amount of triglyceride in a sample and determining an amount of brown adipose tissue or beige fat in the sample based on the data and the amount of triglyceride determined in the first determination step.

6: The analysis method according to claim 5, wherein the PLS regression model is a model based on an intensity of lipid absorption peak in the spectrum data subjected to the noise removing process.

7: The analysis method according to claim 5, wherein the first determination step includes determining whether brown adipose tissue, beige fat, and white fat are present in the sample based on whether there is a water absorption peak in a 900 nm wavelength band in the spectrum data subjected to the noise removing process.

8: The analysis method according to claim 5, wherein the second determination step includes using data indicating a correlation with an amount of triglyceride in a stimulated sample and determining an amount of brown adipose tissue or beige fat in the stimulated sample based on the data and the amount of triglyceride determined in the first determination step.
